(19) United States
(12) Patent Application Publication
(10) Pub. No.: US 2016/0000384 A1
(43) Pub. Date: Jan. 7, 2016

(54) ELASTICITY MEASUREMENT WITH TOMOGRAPHIC IMAGING

(71) Applicant: Siemens Aktiengesellschaft, Muenchen (DE)

(72) Inventors: Peter Gall, Buckenhof (DE); Berthold Kiefer, Erlangen (DE)

(73) Assignee: Siemens Aktiengesellschaft, Muenchen (DE)

(21) Appl. No.: 14/790,209

(22) Filed: Jul. 2, 2015

(30) Foreign Application Priority Data
Jul. 3, 2014 (DE) 102014212944.3

(51) Int. Cl. A61B 5/00; G06T 7/00; A61B 8/00; A61B 8/08; A61B 5/055; A61B 6/00

(52) U.S. Cl. CPC A61B 5/7292; A61B 5/055; A61B 6/541; A61B 8/543; A61B 6/5205; A61B 8/485; G06T 7/0024; G06T 7/0012

(57) ABSTRACT

In a method and apparatus for determining the elasticity of a material of an examination object, which is included in a subarea of an acquired image volume, data from the image volume are acquired with a tomographic imaging method in at least two different time phases during a body movement of the examination object. At least two recorded images are registered with one another. A deformation field of the material included in the subarea of the acquired image volume is determined from the images which are registered with one another. The local elasticity of the material of the image volume included in the subarea of the acquired image volume is determined on the basis of the determined deformation field.

### BACKGROUND OF THE INVENTION

1. Field of the Invention

The invention concerns a method for determining the elasticity of a material of an examination object included in a subarea of an acquired image volume and to an elasticity acquisition apparatus. Furthermore, the invention relates to a tomographic imaging modality, such as a magnetic resonance system, a computed tomography system, an ultrasound system or a positron-emission tomography system. The invention is described below in the example of a magnetic resonance system, without this being restrictive.

2. Description of the Prior Art

In MR elastography (MRE), such as in an elastography using of another imaging modality, an in-vivo measurement of the visco-elastic parameter of the tissue of a patient to be examined is performed. A patient is understood below to mean both a human and an animal. The actual measurement is performed e.g. with magnetic resonance tomography (MRT). The elastography plays a particularly significant role in the differential diagnosis of cancer diseases. Elastography capability will in the future be an important enhancement to conventional methods for determining the mechanical properties of tissue, comparable to palpation.

With elastography, the local hardness (elasticity) of the tissue is concluded indirectly. This indirect procedure is necessitated because non-invasive imaging methods that are currently available are not sensitive to mechanical parameters (elasticity). This indirect procedure is usually realized by coupling mechanical sinusoidal waves into the tissue to be measured, and the simultaneous measurement and diagrammatic reproduction of these waves, e.g. by magnetic resonance tomography (MRT).

The propagation of mechanical waves into complex viscous media (like e.g. tissue) is described physically by a partial differential equation. In such cases, the local properties of a wave are linked to the properties of the wave in the next adjacent element by material constants (density, compressibility, attenuation and elasticity). If the density and compressibility in the tissue are assumed to be constant for instance, the attenuation and the elasticity can be concluded in a known wave propagation with the use of the partial differential equation. In other words, the elastic properties of the tissue influence the wave propagation. It is therefore possible with known (or measured) wave propagation to determine the elastic properties in a computational manner. This relation is well known. In order to be able to determine the coefficients of the differential equation, the dynamic variables of the system (speed and acceleration or delay) have to be measured.

Basically contrast images are recorded in magnetic resonance tomography, which provide information relating to the density of the structure being imaged. Dynamic processes can also be visualized with phase contrast measurements. Images are generated during such a phase contrast measurement, the contrast of which is proportional to the movement of an object.

With MR elastography, a sinusoidal mechanical wave is first applied so as to act on the tissue to be examined, prior to the contrast measurement. The applied mechanical wave produces a periodic oscillation in the tissue (forced oscillation). A special wave generation device is usually used for this purpose. Shortly after producing the oscillations in the body, extremely complex wave propagation phenomena occur within the tissue. In order to simplify the reconstruction, it is useful to start the MR measurement only after a specific waiting time, for instance approximately two seconds after producing the oscillation. After this waiting time, the oscillation is stable, the tissue now oscillates in a sinusoidal manner. A sinusoidal three-dimensional oscillation can be characterized by the amplitude and phase in the respective spatial direction, i.e. in other words six numerical values per point.

Visually expressed, the MR recording (data acquisition) is synchronized with the mechanical wave such that the contrast in the MR image is proportional to the wave. The MR recording is thus used only as a camera, in order to actuate a type of snapshot of the mechanical wave in the tissue. A number of snapshots at different times generate an image sequence, with which the continual propagation of the wave in the tissue can be acquired approximately. This sequence of image recordings is used as the basis of the subsequent reconstruction of the elastic parameters.

The six numerical values mentioned, which characterize the sinusoidal three-dimensional oscillation in the examination object, can be determined with the recorded sequence of wave propagation. The oscillation of the tissue thus can be locally measured MR imaging. In order to calculate the visco-elastic parameters, the differential equation system mentioned above is reverse-calculated so that the coefficients of the differential equation system can be determined.

An image or a type of cards of the region or image volume to be examined can finally be generated from the determined locally dependent visco-elastic parameter values, the respective cards specifying the spatial distribution of the elasticity in the examination region or image volume.

The conventional image recording takes place with a completely still patient, i.e. the recording of the MR images is performed in a breathing pause of the patient, in order to avoid a distortion or disturbance of the acquired MR images due to superimposition of the wave movement generated by the external wave generator in the examination region and movement of the patient. An additional synchronization device or synchronization circuit is required for this purpose, which matches the movement of the patient's body to the measurement sequence.

### SUMMARY OF THE INVENTION

An object of the present invention is to provide an MR elastography method that can be implemented with less outlay than conventionally.

The basis underlying the inventive method is the insight that, without mechanical excitation by a wave generator, the breathing movement or in general terms the natural movement of the body of the patient or of the object to be examined, exhibits dynamic processes that can be identified by acquiring a series of proton density-weighted volume images, and elasticity values for the imaged volume can be determined therefrom. In other words, the invention avoids the use of a wave generator, and instead uses the natural body movement of the patient in order to represent the elasticity in a specific image volume with a dynamic imaging method.

In the method for determining the elasticity of a material of an examination object contained in a subarea of an acquired image volume, for instance an animal or a human, an image volume is firstly recorded e.g. with an MR imaging method in at least two different time intervals during a body movement of the examination object. At least two images of the image volume are thus recorded. An MR imaging method can be used, for instance for the recording. The raw data acquired during the imaging is then reconstructed to form image data in the image space. The images are registered with one another in an additional method step. An image registration is used, for example, to bring two or more images recorded temporally one after the other into concordance as closely as possible. One of the images is defined as a reference image. The remaining images are referred to as object images in this context. In order to adjust these optimally to the reference image, a balancing transformation is calculated. The images to be registered differ from one another because they were recorded at different points in time in different movement states. A deformation field is then determined from the registration of the images that are recorded in a temporally offset manner or the transformation determined in this way. The deformation field provides information relating to the dynamic behavior of the material included in a subarea of the acquired image volume. The deformation field includes information relating to the local or locally-dependent deformation in the subarea of the image volume. It is then possible to conclude the elasticity of the material included in the subarea of the image volume from the determined local deformation by calculating the mentioned differential equation system. The elasticity can only be estimated directly, e.g. by a proportionality assumption. Otherwise the differential equation must be solved, which is possible for instance with the use of a biomechanical model that is subsequently determined.

A simplified measuring set-up without a wave generation facility and without a synchronization circuit is thus enabled with the inventive method.

The inventive elasticity determination device includes an image recording unit (scanner), which is configured to acquire an image volume of an examination object with a tomographic imaging method in at least two different phases during the breathing movement of the examination object. The tomographic imaging method may include, for instance, a time series of proton-density weighted volume images, with which dynamic processes can be made visible. Furthermore, the elasticity determination device includes an image registration unit, which is configured to register the at least two recorded images with one another. The at least two recorded images can be recorded, as noted above, in the state of exhalation and in the state of inspiration. The elasticity determination device has in addition a deformation field determination unit, which is configured to determine a deformation field from the images which have been registered with one another. The deformation field includes the information relating to the local deformation of the material included at least in a subarea of the image volume. The elasticity determination device has an elasticity determination unit, which is configured to determine the elasticity of the material included in the subarea of the image volume on the basis of the determined local deformation. The elasticity is produced in such cases from the relationship between the stress and expansion of a preformed material in the subarea of the image volume. For instance, the elasticity is clearly changed in a subarea of an organ to be examined, by comparison with its surroundings, if a tumor is established there or if the affected tissue is hardened by a fibrosis. This change can be displayed graphically with the use of the described elasticity determination device.

An imaging medical modality in accordance with the invention is a tomographic imaging system and includes the inventive elasticity determination device.

Many of the afore-cited components in the inventive elasticity determination device, in particular the image registration unit, the deformation field determination unit and the elasticity determination unit, can be realized wholly or partially in the form of software modules. This is advantageous because existing hardware facilities can be retrofitted for implementation of the inventive method by a software installation. The invention therefore also encompasses a non-transitory, computer-readable data storage medium that can be loaded directly into a processor of a programmable control computer of an imaging medical modality. The storage medium is encoded with program code (programming instructions), in order to cause all steps of the inventive method to be implemented, when the program is executed in the programmable control computer. The control computer can also include distributed units, for instance an image acquisition unit, an image registration unit, a deformation field determination unit and an elasticity determination unit, etc. or be part of the computer and actuate the aforementioned. A computer terminal can be considered to be a control computer, with which a user can actuate entries in order to control an imaging medical modality.

In a preferred embodiment of the inventive method, the body movement includes a breathing movement. The breathing movement is advantageous because it takes place relatively uniformly and periodically. Furthermore, the natural breathing movement does not need to be generated or triggered by external apparatuses, so that additional means are not required in order to generate mechanical waves in the body or to synchronize the excited mechanical waves.

In an embodiment of the inventive method, a vector field is firstly generated when determining the deformation field on the basis of the registration of the at least two image recordings. The vector field contains a displacement part and a deformation part. These two parts of the vector field are to be attributable to the fact that the body tissue in the image volume to be recorded is both displaced and also deformed in the case of a body movement. The vector field is then divided into a deformation part and a displacement part of the vector field and the deformation field is calculated on the basis of the deformation part of the vector field. In this manner, image artifacts determined conventionally as interference or the image data assigned to these image artifacts can thus be divided into their components such that the information required for the determination of the elasticity of an examination region can be insulated.

In an embodiment of the inventive method, the at least two image recordings are implemented in the breathed-out state and in the breathed-in state. In other words, at least one of the image recordings is recorded at the point in time at which the patient has partially or completely breathed out and at least one of the images recorded is recorded at the point in time at which the patient has partially or completely breathed in. It is to be expected that the change in the physical variables to be measured is at its greatest between these two states and a particularly precise measurement result can thus also be achieved.

In an alternative embodiment of the inventive method, a registration algorithm is used for registration on the basis of a biomechanical model. The biomechanical model models for instance, certain mechanical properties, such as spring constants, from which the elasticity of the region to be examined can be concluded. In this alternative embodiment of the inventive method, the registration step and the step for determining the deformation or elasticity are combined. When applying a biomechanical model, the liver is segmented in all volumes, as a result of which the surface is described. The registration brings the surfaces and the volumes into congruence. The liver tissue can then be modeled, e.g. using FEM (Finite Element Method) with the use of elasticity values from conventional elastography. It is now possible, for instance, to iteratively locally compare whether fibrotic or normal liver tissue suits deformation by registration better.

### BRIEF DESCRIPTION OF THE DRAWINGS

FIG. 1 is a flowchart of an exemplary embodiment of the inventive method.

FIG. 2 schematically illustrates a vector field generated in the inventive method by the image registration.

FIG. 3 schematically illustrates a deformation field separated from the vector field illustrated in FIG. 2.

FIG. 4 is a schematic representation of an inventive elasticity determination apparatus.

FIG. 5 shows a magnetic resonance system as an example of an inventive imaging medical modality according to an exemplary embodiment of the invention.

### DESCRIPTION OF THE PREFERRED EMBODIMENTS

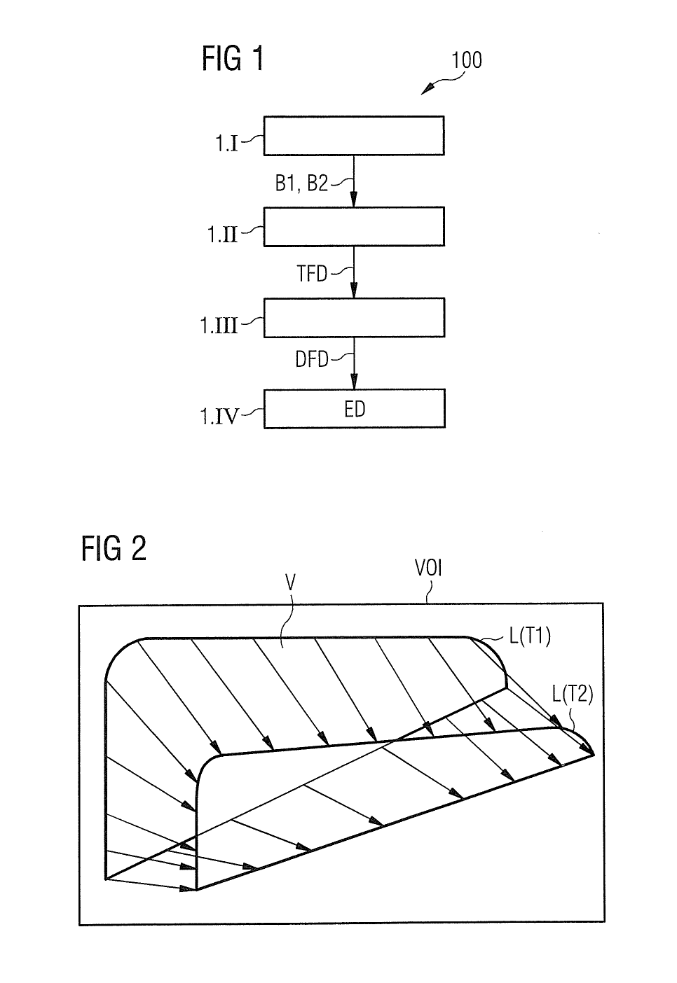

In FIG. 1, the inventive method 100 according to an exemplary embodiment of the invention is shown in a flowchart. The inventive tomographic imaging method is described as an example below as an MR imaging method. Other tomographic imaging methods are to be applied similarly. In step 1.I an image volume VOI is recorded using an MR imaging method in two different time phases during the breathing movement of a patient or an examination object O. The breathing movement can be acquired for instance with an additional sensor, which detects the breathing movement and outputs a control signal, with the aid of which the MR image recording is implemented in synchrony with the breathing movement. In order to be able to determine the dynamic behavior of the tissue located in the image volume VOI, the recording of at least two MR images B1, B2 is required at different time points.

A synchronization of the breathing movement with the image recording is not absolutely necessary. It only enables the optimal use of the breathing movement for the elasticity measurement. Alternatively, it is also possible to dispense with a synchronization and to implement the MR image recordings at time points which are independent of the breathing movement. With a plurality of MR image recordings, even a temporally randomly controlled MR image recording is useful, with which an averaging can be performed so to speak over all phases of the breathing movement.

The MR image recordings can apparently be characterized as snap shots of the moving image volume VOI of the patient O at specific time points, combined together to form a type of film which acquires information relating to the dynamic properties of the material or tissue disposed in the image volume VOI or a subarea.

The at least two recorded images B1, B2 are registered with one another in step 1.II. On account of the breathing movement and the temporally offset recording of the images B1, B2, recorded objects or identifiable sections in the image volume VOI are spatially offset or deformed with the images B1 and B2. The example of a liver L is shown schematically in FIG. 2, said liver having been recorded at different points in time T1 and T2. The two images L(T1) and L(T2) are shown one above the other in FIG. 2. As apparent in FIG. 2, the liver L is locally displaced and deformed in the different points in time. This change is caused by the breathing movement, during which a mechanical force acts on the liver L and both displaces and also deforms this. The displacement and deformation can be mentioned, described and clarified together by a vector field V, also known as transformation field. For clarification purposes some arrows, which represent vectors of the vector field V, are plotted in FIG. 2. For simplicity, only vectors at the edges of the liver are plotted. As already mentioned, attempts are made during the registration to bring the different recorded images of the liver L into the best possible agreement with one another. In order to adjust the different images optimally to one another, a balancing transformation is calculated. This transformation corresponds precisely to the aforementioned vector field V.

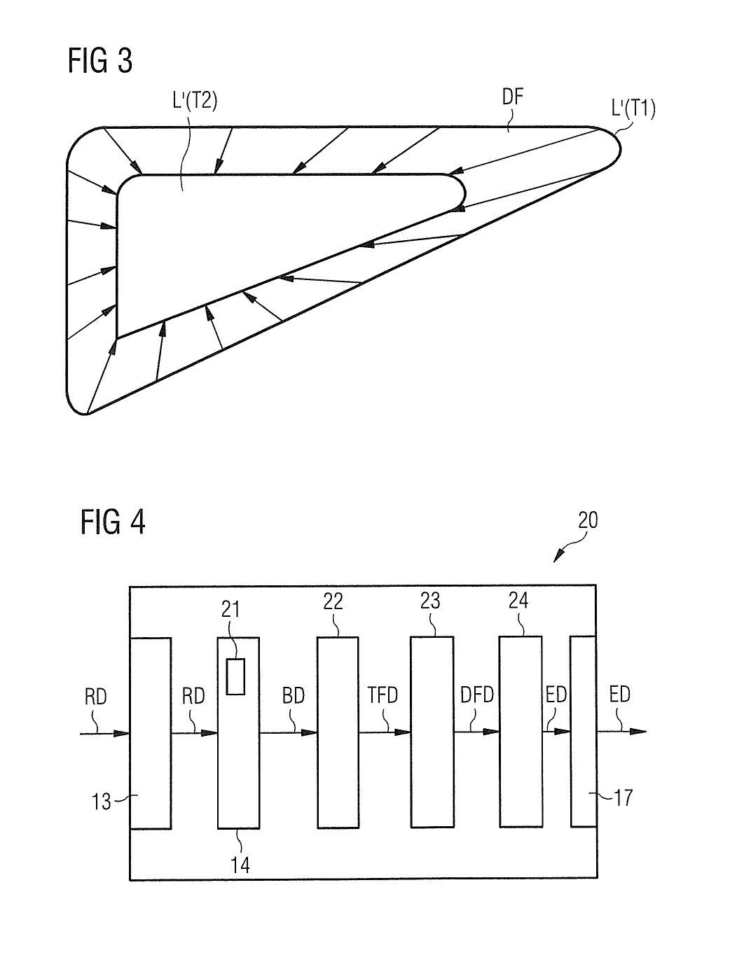

A deformation field DF is determined in step 1.III from the registration of the temporally offset images or on the basis of the transformation field V determined in this way. The deformation field DF is illustrated in FIG. 3. The two images L′(T1) and L′ (T2) of the liver L are shown lying one above the other in FIG. 3. The images L′ (T1) and L′ (T2) are the images L (T1) and L (T2) corrected by the displacement. The deformation field DF characterizes the dynamic behavior in the recorded image volume VOI without displacing the objects recorded in the image volume VOI or taking sections into account. In order to calculate the deformation field DF from the transformation field V, the displacement part and the deformation part of the transformation field V are separated from one another. The deformation part forms the sought-after deformation field DF.

In step 1.IV, the local elasticity E of the image volume is determined on the basis of the determined deformation field DF. When the elasticity is determined, it is sufficient for display purposes if only relative values are estimated. The diagnosis method is only to show the elasticity on the basis of color cards. A surrogate parameter is also acceptable here.

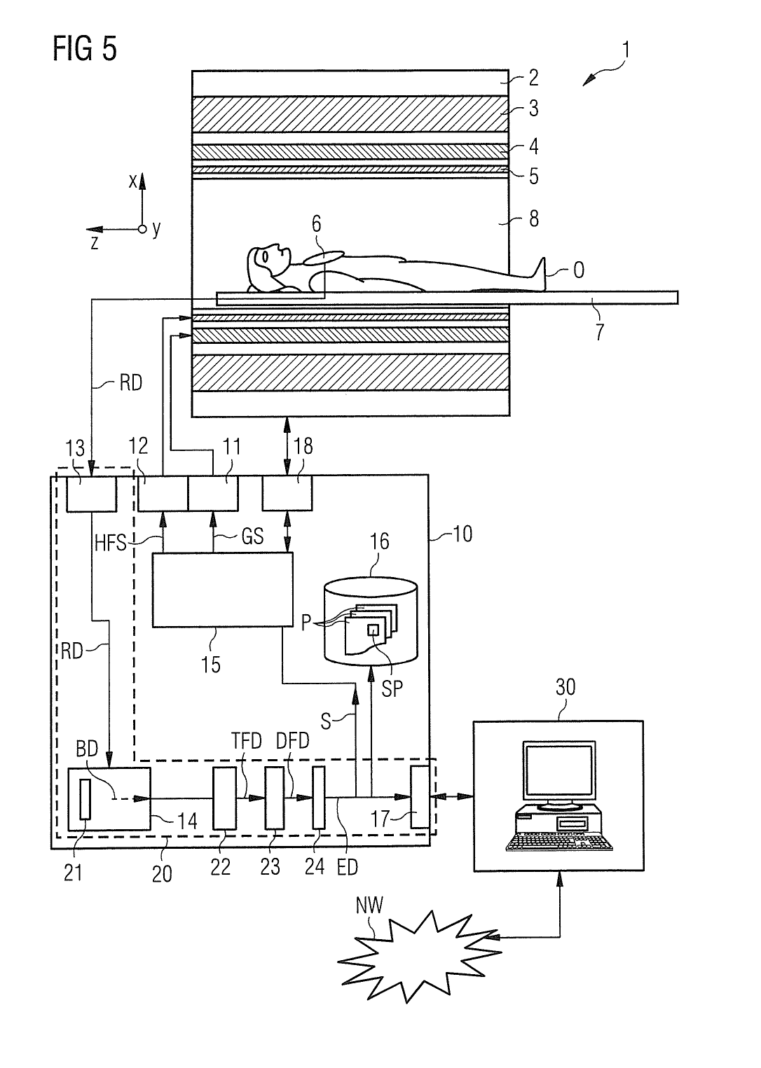

A schematic representation of an inventive elasticity determination facility 20 is shown in FIG. 4. In the exemplary embodiment shown, the elasticity determination facility 20 includes a RF receive unit 13, which receives, demodulates and digitalizes magnetic resonance signals of an MR image recording. The magnetic resonance signals are transferred in digital form as raw data RD to a reconstruction unit 14, which reconstructs the image data BD therefrom. In the exemplary embodiment shown, the reconstruction unit 14 includes another intermediate memory 21, in which reconstructed images are already stored. The RF receive unit 13, the reconstruction unit 14 and the intermediate memory 21 can also be considered to be an image recording unit, in which an image volume VOI of an examination object O with an MR imaging method is acquired in at least two different time phases during the breathing movement of the examination object O. The acquired image data, for instance two images B1 and B2, are forwarded to an image registration unit 22. The image registration unit 22 has the function of registering the recorded images B1, B2 with one another. As mentioned, a vector field V is determined during the registration, which includes a transformation rule for the transformation of one of the images into another. The transformation field data TFD identifying the vector field V is transferred to a deformation field determination unit 23. The deformation field determination unit 23 separates a deformation field DF from the transformation field V, which characterizes the deformation taking place between the recording of the different images B1, B2. The deformation field data DFD are then transferred to an elasticity determination unit 24. A local elasticity E of objects or sections of the image volume VOI is calculated by the elasticity determination unit 24 on the basis of the received deformation field data DFD. The elasticity data ED identifying the local elasticity E is finally transferred to an output interface 17, which forwards the elasticity data ED for instance to a memory or an evaluation unit, like for instance an evaluation terminal.

FIG. 5 shows a schematic representation of an inventively configured magnetic resonance system 1 as an example of an inventive configured imaging medical modality 1. It includes on the one hand the actual magnetic resonance scanner 2 with an examination space 8 or patient tunnel 8 disposed therein. A couch 7 can be guided into this patient tunnel 8, so that a patient O or test person resting thereupon can be supported during an examination at a specific position within the magnetic resonance scanner 2 relative to the magnet system and radio-frequency system arranged therein or can be moved during a measurement between different positions.

Basic components of the magnetic resonance scanner 2 are a basic field magnet 3, a gradient system 4 with magnetic field gradient coils for generating magnetic field gradients in the x-, y- and z-directions and a whole body radio-frequency coil 5. The magnetic field gradient coils in the x-, y- and z-direction can be actuated independently of one another, so that gradients can be applied in any logical spatial directions (for instance in slice selection direction, in phase encoding direction or in read-out direction) by a predetermined combination, wherein these directions generally depend on the selected slice orientation. Similarly, the logical spatial directions can also agree with the x-, y- and z-directions, for instance slice selection direction in the z-direction, phase encoding direction in the y-direction and read-out direction in x-direction. The receipt of magnetic resonance signals induced in the examination object O can take place by way of the whole body coil 5, with which radio-frequency signals are generally also emitted in order to induce the magnetic resonance signals. These signals are, however, usually received with a local coil arrangement 6 with for instance local coils placed on or below the patient O (of which only one is shown here). All of these components are essentially known to those skilled in the art and thus need only be shown schematically in FIG. 5.

The components of the magnetic resonance scanner 2 can be actuated by a control device 10. This may be a control computer, which can be composed of a number of individual computers that may be spatially separated if necessary and connected to one another by suitable cables or the like. This control device 10 is connected to a terminal 30 by way of a terminal interface 17, via which an operator can actuate the entire system 1. In the present case, this terminal 30, as a computer, is equipped with a keyboard, one or a number of monitors and further input devices such as for instance a mouse or suchlike so that a graphical user interface is available to the user.

The control device 10 has inter alia, a gradient control unit 11, which can in turn consist of a number of part components. The individual gradient coils are wired according to a gradient pulse sequence GS with control signals by way of this gradient control unit 11. This is, as described above, gradient pulses, which are set (played out) during a measurement at precisely provided temporal positions and with an accurately predetermined temporal course.

The control device 10 also has a radio-frequency transmit unit 12, in order to feed radio-frequency pulses according to a predetermined radio-frequency pulse sequence RFS of the pulse sequence into the whole body radio-frequency coil 5. The radio-frequency pulse sequence RFS includes, for instance, excitation and refocusing pulses. The receipt of the magnetic resonance signals then takes place with the local coil arrangement 6, and the raw data RD received thereby are read out and processed by an RF receive unit 13. The magnetic resonance signals are transferred in digital form as raw data RD to a reconstruction unit 14, which reconstructs the image data BD therefrom. In the exemplary embodiment shown, the reconstruction unit includes another intermediate memory 21, in which reconstructed images are stored. The acquired image data BD, for instance two images B1 and B2, is forwarded to an image registration unit 22. The image registration unit 22 has the function of registering the recorded images B1, B2 with one another. The transformation field data TFD generated during the registration is transferred to a deformation field determination unit 23. The deformation field determination unit 23 breaks the transformation field V of the transformation field data TFD down into a deformation field DF and a displacement field, wherein the deformation field DF characterizes the deformation taking place between the recording of the various images B1, B2. The deformation field data DFD are then transferred to an elasticity determination unit 24. The elasticity determination unit 24 calculates elasticity data ED of objects or sections of the image volume VOI on the basis of the received deformation field data DFD. The elasticity data ED are then optionally stored in a memory 16 and/or transferred via the interface 17 to the terminal 30 so that the operator can view the data. The elasticity data ED can also be stored and/or indicated and evaluated by way of a network NW at other points.

Alternatively, a radio-frequency pulse sequence can be transmitted by the local coil arrangement and/or the magnetic resonance signals can be received by the whole body radio-frequency coil (not shown), depending on the current switching of the whole body radio-frequency coil 5 and the coil arrangements 6 with the radio-frequency transmit unit 12 or R receive unit 13.

Via a further interface 18, control commands are transferred to other components of the magnetic resonance scanner 2, like e.g. the bed 7 or the basic field magnet 3, or measured values or other information assumed. By moving the bed 7, a desired image volume VOI can be selected, in which certain objects, like for instance organs or special body areas of interest, can be disposed.

The gradient control unit 11, the RF transmit unit 12 and the RF receive unit 13 are actuated in a coordinated manner by a measuring control unit 15. By corresponding commands this provides that the desired gradient pulse sequences GS and radio-frequency pulse sequences RFS are emitted. Furthermore, care must be taken to ensure that the magnetic resonance signals on the local coils of the local coil arrangement 6 are read out and further processed by the RF receive unit 13 at the appropriate point in time. The measuring control unit 15 likewise controls the further interface 18. The measuring control unit 15 can be formed, for instance, from a processor or a number of interacting processors.

The basic execution of such a magnetic resonance measurement and the cited components for actuation are known to those skilled in the art so that they need not be explained in further detail herein. Incidentally, such a magnetic resonance scanner 2 and the associated control facility can still comprise a number of further components which are not explained in detail here. Reference is made at this point to the fact that the magnetic resonance scanner 2 can also be structured differently, for instance with a laterally open patient compartment, or as a smaller scanner, in which only one body part can be positioned. Other tomographic imaging systems such as a computed tomography system or an ultrasound system and positron emission tomography devices, which can be used for the inventive method, are basically also known and are therefore not described in further detail herein.

In order to start a measurement, an operator can usually select a control protocol P provided for this measurement from a memory 16 by way of the terminal 30, in which a plurality of control protocols P are stored for various measurements. The operator can also call up control protocols, for instance from a manufacturer of the imaging medical modality, via a network NW, and modify and if necessary use these protocols.

The measurement course can additionally be synchronized with a breathing movement of the patient. Measurement data relating to the breathing movement of the patient can be acquired here for instance by the interface 18 and can be used to clock the measurement by the measurement control unit 15.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for determining elasticity of a material of an examination object, comprising:
operating a tomographic imaging scanner to acquire images of a volume of an examination object situated in the tomographic imaging scanner, respectively during at least two different time phases of a body movement of the examination object, said volume including material having an elasticity;
providing said images to a processor and, in said processor, bringing said images into registration with each other;
in said processor, determining a deformation field of said material from said images that are in registration with each other;
in said processor, determining a local elasticity of said material from said deformation field; and
from said processor, emitting an electronic signal representing said local elasticity at an output of said processor.

2. A method as claimed in claim 1 wherein said body movement is breathing movement.

3. A method as claimed in claim 2 comprising acquiring one of said images when said examination object is in an exhaled state and acquiring another of said images when said examination object is in an inhalation state.

4. A method as claimed in claim 1 comprising, in said processor, generating a vector field when said deformation field is determined from said images in registration with each other.

5. A method as claimed in claim 4 comprising, in said processor, deconstructing said vector field into a deformation portion and a displacement portion, and calculating said deformation field from said deformation portion of said vector field.

6. A method as claimed in claim 1 comprising bringing said images into registration with each other by executing a registration algorithm in said processor based on a biomedical model for registration.

7. An elasticity determination apparatus, comprising:
a tomographic imaging scanner;
a control computer configured to operate said tomographic imaging scanner to acquire images of a volume of an examination object situated in the tomographic imaging scanner, respectively during at least two different time phases of a body movement of the examination object, said volume including material having an elasticity;
said control computer being configured to bring said images into registration with each other;
said control computer being configured to determine a deformation field of said material from said images that are in registration with each other;
said control computer being configured to determine a local elasticity of said material from said deformation field; and
said control computer being configured to emit an electronic signal representing said local elasticity at an output of said control computer.

8. An elasticity determination apparatus as claimed in claim 7 wherein said tomographic imaging scanner is a scanner selected from the group consisting of a magnetic resonance scanner, a computed tomography scanner, an ultrasound scanner, and a positron-emission tomography scanner.

9. A non-transitory, computer-readable data storage medium encoded with programming instructions, said storage medium being loaded into a control computer of a tomographic imaging system, said tomographic imaging system comprising a tomographic imaging scanner, and said programming instructions causing said control computer to:
operate said tomographic imaging scanner to acquire respective images of a volume of an examination object situated in the tomographic imaging scanner, during at least two different time phases of a body movement of the examination object, said volume including material having an elasticity;
bring said images into registration with each other;
determine a deformation field of said material from said images that are in registration with each other;
determine a local elasticity of said material from said deformation field; and
emit an electronic signal representing said local elasticity at an output of said control computer.
